(19) United States
(12) Patent Application Publication
(10) Pub. No.: US 2012/0143295 A1
(43) Pub. Date: Jun. 7, 2012

(54) Menstrual Relief Band

(76) Inventor: Brad Lee St. John Moore, Glendale, AZ (US)

(21) Appl. No.: 12/959,366

(22) Filed: Dec. 3, 2010

(51) Int. Cl. A61F 7/08

(52) U.S. Cl. 607/112

(57) ABSTRACT

A band that employs low voltage resistance wire (11) to provide heat for the purpose of relieving the pain and discomfort caused by menstrual cramps. It is wrapped around the hip/lower abdominal area of the body and velcro (1, 2) is used to hold it in place. In addition to the velcro, elastic materials (7) enable the abdominal heat band to have an adjustable size so that it is able to fit both large and small individuals. Elastic also helps provide a snug fit allowing the user to effectively conceal the band beneath their clothing if desired. A remote control (3), which also houses the battery, controls the power and intensity of the heat. The remote is detachable from the band which allows the band to be washed without risk of getting the remote control wet.

### CROSS-REFERENCE TO RELATED APPLICATIONS

This application claims the benefit of provisional patent application Ser. No. 61/266117, filed 2009 Dec. 2 by the present inventor.

### FEDERALLY SPONSORED RESEARCH

None.

### SEQUENCE LISTING

None.

### BACKGROUND

Heating pads have long been used to help relieve the pain and discomfort caused by sore muscles. However, heating pads are a largely immobilizing method of applying heat to oneself as they generally require being within reach of an electrical outlet. They also require enough electricity to pose the risk of electric shock should any of the wiring become exposed. Furthermore, few individuals are comfortable with being seen publicly using therapeutic devices, so using a heating pad where the individual can be seen by other people (such as at work) is an undesirable prospect.

These are all problems for women who suffer from painful menstrual cramps. These cramps are caused by the blood supply to the uterus being constricted while the muscles in the lower abdominal area contract. The application of heat to this area is often used to treat this pain because it increases blood-flow and reduces muscle stiffness, thus effectively soothing the pain without any need for medication.

U.S. Pat. No. 5,928,275 to Yates (1999) demonstrates the design for a belt-like, body heating apparatus. The belt employs portable methods of heating that focuses primarily on the distribution of said heat throughout the entire body by means of warming chemicals (one time use liquid/gel packets that heat up for a certain amount of time) being placed in pouches on the belt.

U.S. Pat. No. 5,398,667 to Witt (1995) and U.S. Pat. No. 4,470,417 to Gruber (1984) both disclose methods of applying heat to the lower back Similarly to Yates, Witt proposes using chemical means of creating heat. This can be considered both wasteful and incontinent, as in order to continue using this method, the chemical packets must be replaced every time the devices are used.

None of these are specifically designed to treat the pain caused by menstrual cramps, even if they could be used to do so (as could many heating devices). One problem with using multi-purpose devices is that they are designed primarily to heat larger portions of the body and menstrual cramps are treated by applying heat to only a very small area. This results in an excess of resistance wire being used, thus greatly increasing the amount of power consumed. Additionally, having heat applied over an undesired area of the body also increases perspiration in those areas (and over-all body temperature), leading to a decrease in comfort and hygiene over time.

U.S. Pat. No. 4,628,930 to Williams (1985) and U.S. Pat No.7,824,437 to Saunders (2010) both disclose methods of heating specifically for the purpose of relieving pain caused by menstrual cramps. They suggest that resistance wire can be concealed within a panty so that it can be worn unnoticed and apply heat directly to the area, but many women would be discouraged from using this due to the inconvenience in needing to remove their pants and undergarments in order to remove or put on the panty.

There is also a lack of ability to adjust the size of the panty design. A single size will fit a small range of women, but for the most part, the panty would have to be constructed in many different sizes in order to fit both small and largely built women. Another downfall of the panty design is that due to its hygienic nature, it would need to be washed frequently regardless of the aqueous environments it was designed to withstand. It should also be noted that the panty would need to extend higher in the front (closer to the naval) than a common, non-therapeutic panty would need to due to the fact that the waistband of said common panty does not rest over the muscles in the lower abdomen and thus, a commonly designed panty would not be able to apply heat to entirety of the appropriate area. The increase in panty size (in the front and/or elsewhere) results in an oversized panty, a further discouragement for use by the average woman.

It is suggested in Williams' design that connecting or disconnecting the battery is how the user controls the power to the heating unit in the panty design, which severely limits when the user would be able to turn it off and on given the location of said battery. This can also be considered a safety hazard given that if the unit becomes too hot for the user, combined with the fact that it is not easily removed, it would be difficult and possibly embarrassing (if in public, for example) for the user to quickly remove the unit or disconnect its power.

### SUMMARY

The menstrual relief band is concealable due to the form-fitting elastic materials from which it is comprised. It fits like a belt and is wrapped around the lower abdominal area of the body so that it crosses over the muscles that are responsible for menstrual cramps. As a result, it is very easy to remove and put on without the need to remove any articles of clothing. In addition, when the battery power is depleted, the entire menstrual relief band or just the remote/battery can easily be removed and recharged without any need to remove additional articles of clothing.

The menstrual relief band is worn either directly against the skin or it can be positioned over the top of the user's underwear or other clothing, fitting snugly and unseen beneath everyday clothing if so desired. Due to the use of velcro (or other adjustable means of attachment) and elastic in its design, it is able to fit both slim and larger women. Furthermore, unlike designs that encompass multiple uses for the heat (such as back pain relief, etc.), the menstrual relief band is able to use less rigid, less thick, and more breathable methods of construction. This results in a design that is form-fitting and easily concealable. Since the menstrual relief band is so light weight, it can be worn for extended periods of time without discomfort.

In addition, the menstrual relief band requires washing far less often than a panty design would because of its more hygienic nature, and it would also require washing less often than bulkier (multi-purpose) design would because of its light weight, breathable material, and decreased surface area. The decrease in surface area (the smaller design) is important because more skin being covered by material results in a greater amount of perspiration, which leads to a decrease in comfort and hygiene. Other, multi-purpose designs also employ an increased amount of resistance wire in order to spread heat over a larger area which is necessary in the relief of back pain. If used to sooth menstrual cramps, however, the user would find that a greater than desired area of the abdomen is being heated, both consuming more power and resulting in an additional decrease in comfort. However, the menstrual relief band specifically targets the muscles responsible for painful menstrual cramps, and as a result, only a specific amount of resistance wire and material is used which makes for a very thin, flexible, and lightweight design.

Heat is provided via low voltage resistance wire which draws its power from a portable battery source. The electricity consumed by the low voltage resistance wire is much less than the electricity consumed by standard heating pads, and as a result, not only is it much safer to operate because the risk of electric shock is removed, but smaller battery packs can be used making the entire unit less uncomfortable and easier to conceal. Low voltage resistance wire is also far more flexible than standard resistance wire, which is important when considering that the menstrual relief band is concealable and form-fitting.

The temperature of the menstrual relief band can easily be adjusted via a small remote control (that also houses the battery power source). The user can select what heat level they prefer and also turn the unit on and off without being required to find a private location to do so in. This is possible because the remote control is separable from the menstrual relief band and is easily attachable to the waistband of the user's pants or the menstrual relief band itself.

### DRAWINGS

FIG. 1 is a front view (the side that faces away from the body of the user) of the menstrual relief band.

FIG. 2 is a back view (the side that is placed against the skin of the user) of the menstrual relief band.

FIG. 3 is a close view of the heating unit, showing basic construction by layers.

FIG. 4 and FIG. 5 show how the menstrual relief band is worn and positioned on the female body from different perspectives.

### DETAILED DESCRIPTION

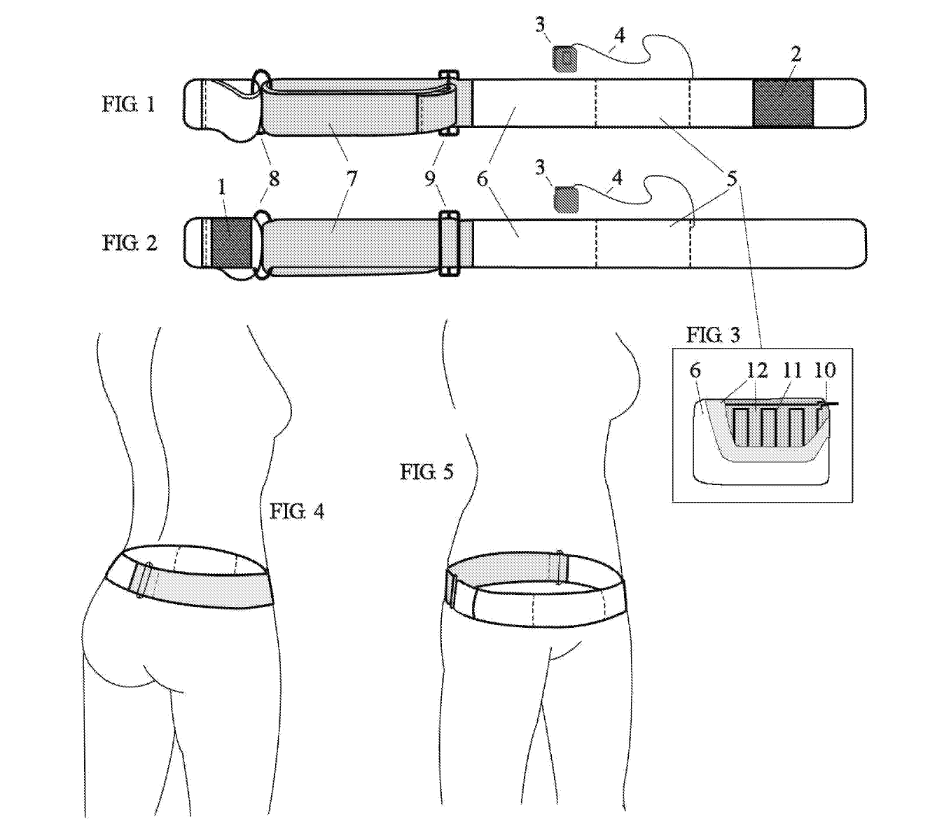

FIG. 1 illustrates the outer side of the band that faces away from the skin of the user. In this application, the hook side of velcro (2) attaches to the loop side of velcro (1) and secures the menstrual relief band to provide a snug and thus concealable and comfortable fit. A material length adjustment ring (9) provides means for lengthening or shortening the size of the menstrual relief band's elastic portion of the material (7) and thus its overall size. A ring (8) is fixed through the loop in material that is created by the adjustment ring (9). To this ring (8), a small tab of material (6) with velcro (2) is attached. The ring (8) is employed to make sure that velcro (1) is always in the same position regardless of any adjustments the user may make to the size of the menstrual relief band. The remote control (3) is attached to the menstrual relief band via a cord (4) that provides power and control to the heating unit (5). Attached to the remote control are means of enabling the user to easily attach the remote control (3) to the waistband of the user's pants or the menstrual relief band itself. The heating unit (5) shown is described in detail under FIG. 3. The combination of material (6) and elastic material (7) enables the menstrual relief band to better conform to the user's body while still staying comfortable. This is important because it adds to the ability of the menstrual relief band to be concealed, and it also focuses the heat inwards to the body instead of outwards where it is wasted. There are multiple materials, elastic and inelastic, employed to compliment each other in order to enhance comfort by allowing more air flow and less friction than a purely elastic material could provide.

FIG. 2 illustrates the side of the band that faces inwards, towards the skin of the user and is the reverse of the side shown in FIG. 1.

FIG. 3 illustrates a general design for the heating unit (5) part of the menstrual relief band. The power cord (4) is detachable via the connector (10) so that if the unit is washed, none of the components in the remote control/battery compartment (3) will get wet. Under a layer of material (6) that covers the heating unit (5), there is a layer of material (12) that is used to insulate the resistance wire (11). This layer of insulation (12) helps distribute heat over an area evenly as well as provide a small amount of padding for wire protection and comfort reasons. It also protects the user from the coming into direct contact with the resistance wire (11) and the waterproof qualities of the insulation prevent said wiring from getting wet due to rain, perspiration, and so on.

FIG. 4 illustrates a rear diagonal view of how the menstrual relief band is worn on the female body.

FIG. 5 illustrates a front diagonal view of how the menstrual relief band is worn on the female body.

### Reference Numerals

1 velcro, hook side

2 velcro, loop side

3 remote control and battery compartment

4 cord

5 heating unit

6 material

7 elastic material

8 tab ring

9 adjustment ring

10 cord connection

11 resistance wire

12 insulation layer

### Operation

The user wraps the menstrual relief band around their lower abdomen. After it is in position, it is tightened comfortably so that it stays in position. The user at this point adjusts their clothing to fit naturally over the menstrual relief band so that it is concealed from view. The controls are then used to power on the unit and set the desired level of heat. After this, the remote may be either clipped to the waistband of the user's pants or to the menstrual relief band itself. If the user prefers, the menstrual relief band may also be worn over the top of the user's clothing.

It is appreciated that numerous changes and modifications may be made to the design of this invention without deviating from the scope of the invention as described.

I claim:

1. A band of material, comprising:
a. elongated elastic means of being formfitting to the user's lower abdominal area in such a way that the band is securable and sizeable using means of attaching one end of the band to the other once it is wrapped around the user and is positioned over the muscles responsible for menstrual cramp pain in said lower abdominal area of the human body,
b. resistance wire that administers adjustable levels of heat, positioned on the area of the band that is worn in such a way that said resistance wire is positioned over said muscles, while means for providing power to said resistance wire are portable,
c. an area of the band no larger than what is approximately required to efficiently and effectively provide heat to said muscles is covered by said resistance wire,
whereby said band of material will apply heat to the muscles responsible for causing the pain associated with menstrual cramps, thus easing said pain.
